(19) United States
(12) Patent Application Publication
(10) Pub. No.: US 2018/0028153 A1
(43) Pub. Date: Feb. 1, 2018

(54) ULTRASOUND DIAGNOSTIC APPARATUS AND ULTRASOUND IMAGING METHOD

(71) Applicant: Toshiba Medical Systems Corporation, Otawara-shi (JP)

(72) Inventors: Koji KUROIWA, Fuchu (JP); Tetsuya KAWAGISHI, Nasushiobara (JP); Takeshi FUKASAWA, Nasushiobara (JP)

(73) Assignee: Toshiba Medical Systems Corporation, Otawara-shi (JP)

(21) Appl. No.: 15/660,576

(22) Filed: Jul. 26, 2017

(30) Foreign Application Priority Data
Jul. 26, 2016 (JP) 2016-146637
Jul. 25, 2017 (JP) 2017-143968

(51) Int. Cl. A61B 8/08; G01S 15/89

(52) U.S. Cl. CPC A61B 8/481; G01S 15/895; G01S 15/8963; G01S 7/52038

(57) ABSTRACT

An ultrasound diagnostic apparatus according to an embodiment includes an ultrasound probe and processing circuitry. The ultrasound probe sequentially executes sets of ultrasound transmission respectively along a plurality of scan lines, the sets each being composed only of transmission of first ultrasound having a first phase and transmission of second ultrasound having a second phase that is substantially 90 degrees different from the first phase. With respect to a first echo signal and a second echo signal that have been obtained via the ultrasound probe and correspond to the first ultrasound and the second ultrasound, respectively, the processing circuitry generates a subtraction signal by subtracting the second echo signal from the first echo signal. The processing circuitry generates an ultrasound image based on the subtraction signals generated for the scan lines.

### CROSS-REFERENCE TO RELATED APPLICATIONS

This application is based upon and claims the benefit of priority from Japanese Patent Application No. 2016-146637, filed on Jul. 26, 2016, and Japanese Patent Application No. 2017-143968, filed on Jul. 25, 2017; the entire contents of which are incorporated herein by reference.

### FIELD

Embodiments described herein relate generally to an ultrasound diagnostic apparatus and an ultrasound imaging method.

### BACKGROUND

Conventionally, a one-dimensional ultrasound probe (also referred to as “1D array probe”) having a plurality of transducer elements arrayed in one line or a two-dimensional ultrasound probe (also referred to as “2D array probe”) having a plurality of transducer elements arrayed in a matrix has been used in an ultrasound diagnostic apparatus. A 2D array probe has a larger number of channels than a 1D array probe and is therefore capable of acquiring images with higher spatial resolution than a 1D array probe and rotating a scanned section with the position of the probe being fixed.

In general, the number of channels of a 2D array probe is higher than the number of channels of the main body side of the apparatus. For this reason, an approach is taken in which the transducer elements are divided into groups called sub-arrays, and acquired signals are synthesized (the delays thereof are added up) with respect to each of the groups, so that the number channels used for transmission from the 2D array probe to the main body side of the apparatus is reduced. With this approach, however, noise might be caused when the signals are synthesized.

### BRIEF DESCRIPTION OF THE DRAWINGS

FIG. 1 is a block diagram illustrating an exemplary configuration of an ultrasound diagnostic apparatus according to an embodiment;

FIG. 2 is a block diagram illustrating an exemplary configuration of B-mode processing circuitry illustrated in FIG. 1;

FIG. 3A and FIG. 3B are diagrams for explaining the configuration of an ultrasound probe illustrated in FIG. 1;

FIG. 4 is a diagram for explaining an imaging method employed in the ultrasound diagnostic apparatus according to the embodiment;

FIG. 5 is a flowchart for explaining a processing procedure employed in the ultrasound diagnostic apparatus according to the embodiment;

FIG. 6 is a diagram for explaining effects that are produced by the ultrasound diagnostic apparatus according to the embodiment;

FIG. 7 is a flowchart for explaining a processing procedure employed in an ultrasound diagnostic apparatus according to another embodiment; and

FIG. 8 is a block diagram illustrating an exemplary configuration of the ultrasound diagnostic apparatus according to still another embodiment; and

FIG. 9 is a block diagram illustrating an exemplary configuration of the ultrasound diagnostic apparatus according to still another embodiment.

### DETAILED DESCRIPTION

Embodiments are aimed at providing an ultrasound diagnostic apparatus and an ultrasound imaging method that are capable of removing noise.

An ultrasound diagnostic apparatus according to an embodiment includes an ultrasound probe and processing circuitry. The ultrasound probe sequentially executes sets of ultrasound transmission respectively along a plurality of scan lines, the sets each being composed only of transmission of first ultrasound having a first phase and transmission of second ultrasound having a second phase that is substantially 90 degrees different from the first phase. With respect to a first echo signal and a second echo signal that have been obtained via the ultrasound probe and correspond to the first ultrasound and the second ultrasound, respectively, the processing circuitry generates a subtraction signal by subtracting the second echo signal from the first echo signal. The processing circuitry generates an ultrasound image based on the subtraction signals generated for the scan lines.

The following describes ultrasound diagnostic apparatuses and ultrasound imaging methods according to embodiments with reference to the drawings.

### Embodiment

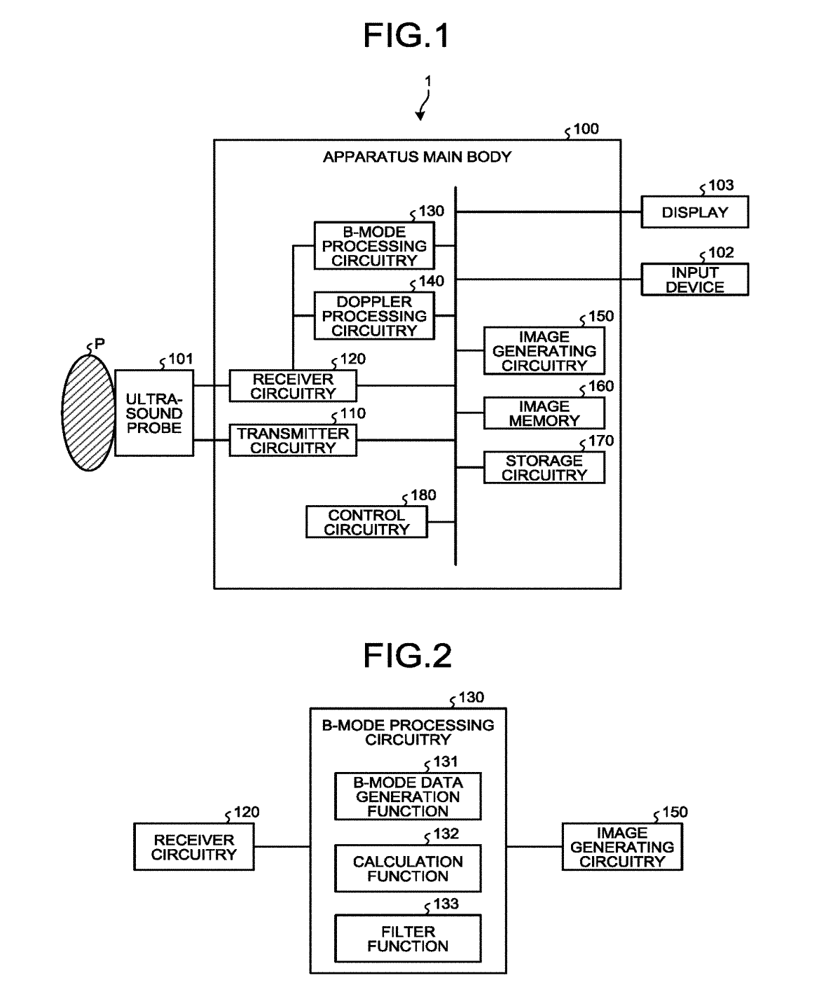

FIG. 1 is a block diagram illustrating an exemplary configuration of an ultrasound diagnostic apparatus 1 according to an embodiment. As illustrated in FIG. 1, the ultrasound diagnostic apparatus 1 according to the embodiment includes an apparatus main body 100, an ultrasound probe 101, an input device 102, and a display 103. The ultrasound probe 101, the input device 102, and the display 103 are individually connected to the apparatus main body 100.

The ultrasound probe 101 includes a plurality of transducer elements (piezoelectric transducer elements). The ultrasound probe 101 is brought into contact with a body surface of a subject P and transmits and receives ultrasound (performs ultrasound scanning). The transducer elements generate ultrasound based on drive signals supplied from transmitter circuitry 110 included in the apparatus main body 100 to be described later. The generated ultrasound is reflected by a surface where acoustic impedance is discontinuous inside the subject P, and received as reflected wave signals (received echoes) by the transducer elements. The ultrasound probe 101 transmits the reflected wave signals received by the transducer elements to the transmitter circuitry 110.

While this embodiment describes a case in which the ultrasound probe 101 is a two-dimensional ultrasound probe (also referred to as “2D array probe”) having a plurality of transducer elements arrayed in a matrix (in a grid), this is not a limiting case. For example, the ultrasound probe 101 may be a one-dimensional ultrasound probe (also referred to as “1D array probe”) having a plurality of transducer elements one-dimensionally arrayed in a certain direction.

The input device 102 includes a mouse, a keyboard, a button, a panel switch, a touch command screen, a foot switch, a track ball, a joystick, or the like, and receives various setting requests from an operator of the ultrasound diagnostic apparatus 1 and transfers the received various setting requests to the apparatus main body 100.

The display 103 displays thereon a graphical user interface (GUI) through which the operator of the ultrasound diagnostic apparatus 1 inputs various setting requests using the input device 102, and displays thereon data such as ultrasound image data generated in the apparatus main body 100.

The apparatus main body 100 is an apparatus that generates ultrasound image data based on reflected wave signals received by the ultrasound probe 101. As illustrated in FIG. 1, the apparatus main body 100 includes, for example, the transmitter circuitry 110, receiver circuitry 120, B-mode processing circuitry 130, Doppler processing circuitry 140, image generating circuitry 150, an image memory 160, storage circuitry 170, and control circuitry 180. The transmitter circuitry 110, the receiver circuitry 120, the B-mode processing circuitry 130, the Doppler processing circuitry 140, the image generating circuitry 150, the image memory 160, the storage circuitry 170 and the control circuitry 180 are communicably connected to one another. The transmitter circuitry 110, the receiver circuitry 120, the B-mode processing circuitry 130, the Doppler processing circuitry 140, the image generating circuitry 150 and the control circuitry 180 are an example of processing circuitry.

The transmitter circuitry 110 includes components such as a pulser circuit. The pulser circuit repeatedly generates a rate pulse for forming, at a certain rate frequency (pulse repetition frequency (PRF)), ultrasound to be transmitted and outputs the generated rate pulse to the ultrasound probe 101.

The transmitter circuitry 110 is controlled by the control circuitry 180 to output the value of amplitude of a drive signal output by a pulser 33, to be described later, to a transmission/reception control circuit 31 to be described later. The transmitter circuitry 110 is controlled by the control circuitry 180 also to output a delay amount of a reflected wave signal in a delay adding circuit 37 to be described later.

The receiver circuitry 120 includes an analog-to-digital (A/D) converter and a received-beam former. Upon receiving reflected wave signals output from the ultrasound probe 101, the receiver circuitry 120 first uses the A/D converter to convert the reflected wave signals into digital data and then uses the received-beam former to: perform phasing addition processing on these pieces of digital data from respective channels to generate reflected wave data; and transmit the generated reflected wave data to the B-mode processing circuitry 130 and the Doppler processing circuitry 140.

The B-mode processing circuitry 130 receives the reflected wave data output from the receiver circuitry 120, and performs processing such as logarithmic amplification and envelope detection on the received reflected wave data to generate data (B-mode data) in which signal intensities are represented by brightness of luminance.

FIG. 2 is a block diagram illustrating an exemplary configuration of the B-mode processing circuitry illustrated in FIG. 1. As illustrated in FIG. 2, the B-mode processing circuitry 130 includes a B-mode data generation function 131, a calculation function 132, and a filter function 133. The B-mode data generation function 131 performs processing such as logarithmic amplification, envelope detection, and logarithmic compression on reflected wave data to generate B-mode data. When ordinary B-mode imaging is being performed, the calculation function 132 and the filter function 133 do not execute any processing, so that the B-mode data generation function 131 generates B-mode data from reflected wave data received from the receiver circuitry 120. Otherwise, when the calculation function 132 and the filter function 133 execute processing, the B-mode data generation function 131 generates B-mode data from data output by the filter function 133. The processing to be executed by the calculation function 132 and the filter function 133 is described later.

The Doppler processing circuitry 140 receives reflected wave data output from the receiver circuitry 120, performs frequency analysis on the received reflected wave data to obtain velocity information therefrom, extracts echo components of a blood flow, tissue, and a contrast medium that are based on the Doppler effect, and generates data (Doppler data) obtained by extracting moving object information such as an average velocity, a distribution, and power at multiple points.

The image generating circuitry 150 generates ultrasound image data from the data generated by the B-mode processing circuitry 130 and the Doppler processing circuitry 140. The image generating circuitry 150 generates, from the B-mode data generated by the B-mode processing circuitry 130, B-mode image data in which the intensities of reflected waves are represented by luminance. The image generating circuitry 150 also generates Doppler image data representing information on the moving body from the Doppler data generated by the Doppler processing circuitry 140. Doppler image data is velocity image data, distribution image data, power image data, or image data obtained by combining any of the foregoing data.

Typically, the image generating circuitry 150 converts (scan-converts) a scan-line signal string of ultrasound scanning into a scan line signal string of a video format represented by a television, for example, and generates ultrasound image data for display. Specifically, the image generating circuitry 150 generates the ultrasound image data for display by performing coordinate transform according to a mode used by the ultrasound probe 101 for ultrasound scanning. In addition to the scan-converting, the image generating circuitry 150 performs, as various types of image processing, image processing (smoothing processing) for regenerating a luminance-value averaged image using a plurality of scan-converted image frames, or image processing (edge enhancement processing) using a differential filter within an image, for example. The image generating circuitry 150 synthesizes the ultrasound image data with text information on various parameters, a scale, a body mark, and the like.

The image memory 160 is a memory that stores therein image data (such as B-mode image data and Doppler image data) generated by the image generating circuitry 150. The image memory 160 can store therein data generated by the B-mode processing circuitry 130 and the Doppler processing circuitry 140. The B-mode data and the Doppler data stored in the image memory 160 can be called up, for example, by the operator and are processed by the image generating circuitry 150 into the ultrasound image data for display.

The storage circuitry 170 stores therein: control programs for performing transmission and reception of ultrasound, image processing, display processing, and the like; and various kinds of data such as diagnostic information (such as patient identification data (ID) and findings of a doctor), diagnostic protocols, and various body marks. The storage circuitry 170 is also used for archiving data stored in the image memory 160 as needed. Data stored in the storage circuitry 170 can be transferred to an external device via an interface unit (not illustrated).

The control circuitry 180 controls the entire processing in the ultrasound diagnostic apparatus 1. Specifically, based on various setting requests input by the operator through the input device 102 and various control programs and various kinds of data read from the storage circuitry 170, the control circuitry 180 controls processing in components such as the transmitter circuitry 110, the receiver circuitry 120, the B-mode processing circuitry 130, the Doppler processing circuitry 140, and the image generating circuitry 150. The control circuitry 180 causes the display 103 to display thereon ultrasound image data stored in the image memory 160.

The transmitter circuitry 110, the receiver circuitry 120, the B-mode processing circuitry 130, the Doppler processing circuitry 140, the image generating circuitry 150, and the control circuitry 180 that are embedded in the apparatus main body 100 may each be constructed of hardware of a processor (such as a central processing unit (CPU), a micro-processing unit (MPU), or an integrated circuit).

For example, the storage circuitry 170 has processing functions to be executed by the respective processors recorded therein in the form of computer executable programs. That is, the respective processors read out computer programs from the storage circuitry 170 and executes the computer programs, thereby implementing functions corresponding to the respective computer programs. For example, the B-mode data generation function 131, the calculation function 132, and the filter function 133 illustrated in FIG. 2 are functions to be implemented with computer programs corresponding to the respective processing functions read out from the storage circuitry 170 and executed by the B-mode processing circuitry 130. In other words, the B-mode processing circuitry 130 includes the functions illustrated in the B-mode processing circuitry 130 in FIG. 2 only when having read out the respective computer programs.

FIG. 1 and FIG. 2 illustrates that the only one B-mode processing circuitry 130 is provided to implement the processing functions to be executed by the B-mode data generation function 131, the calculation function 132, and the filter function 133. However, processing circuitry may be obtained as a combination of a plurality of independent processors, and may have the respective functions implemented with the computer programs executed by the respective processors.

Next, the ultrasound probe 101 connected to the apparatus main body 100 in this embodiment is described. In this embodiment, the ultrasound probe 101 is a 2D array probe having a plurality of transducer elements arrayed two-dimensionally in a matrix.

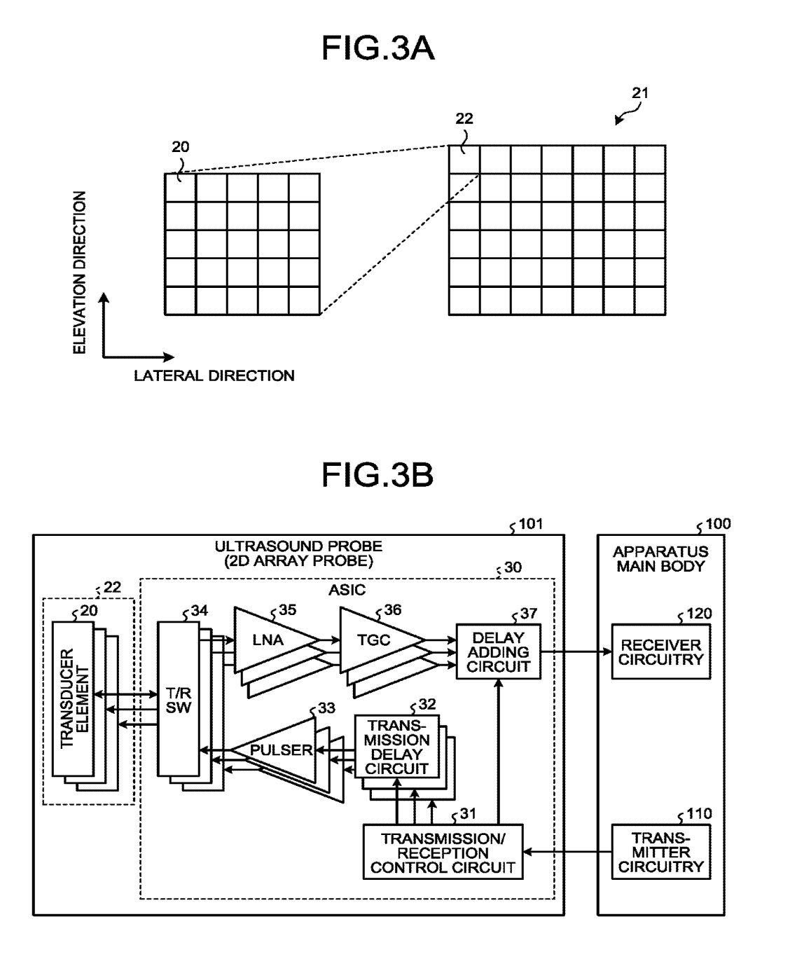

FIG. 3A and FIG. 3B are diagrams for explaining the configuration of the ultrasound probe 101 illustrated in FIG. 1. FIG. 3A illustrates the arrangement of a plurality of transducer elements included in the ultrasound probe 101 that is a two-dimensional array probe. FIG. 3B illustrates an exemplary configuration of the internal configuration of the ultrasound probe 101. Illustrations of FIG. 3A and FIG. 3B are merely examples, and any conventional two-dimensional array probe may be adopted.

As illustrated in FIG. 3A, the ultrasound probe 101 is a 2D array probe having a plurality of transducer elements 20 (also referred to as “transducer element group”) arrayed in lines extending in the lateral direction and in lines extending in the elevation direction. In the 2D array probe, all of the transducer elements 20 included in the ultrasound probe 101 is referred to as a main array 21. The main array 21 is divided into a plurality of sub-arrays 22 in a lateral direction and an elevation direction. For example, the sub-arrays 22 are grouping such that the transducer elements 20 corresponding to the main array 21 are grouped into a certain number of transducer elements 20. In the example of FIG. 3A, the sub-array 22 includes twenty-five transducer elements 20 in which five lines each including five transducer elements 20 arranged side by side in the lateral direction are arranged side by side in the elevation direction. In other words, the sub-array 22 includes 25 transducer elements 20 arranged in a 5-by-5 matrix. In the example of FIG. 3A, only one transducer element is indicated as the transducer element 20 while the others are not indicated as the transducer elements 20. Similarly, only one sub-array is indicated as the sub-array 22 while the others are not indicated as the sub-arrays 22.

As illustrated in FIG. 3B, the ultrasound probe 101 includes an application specific integrated circuit (ASIC) 30. The ultrasound probe 101 further includes, on the ASIC 30, electronic circuits that function as the transmission/reception control circuit 31, transmission delay circuits 32, the pulsers 33, transmission/reception switches (T/R SWs) 34, low noise amplifiers (LNAs) 35, time gain controllers (TGCs) 36, and a delay adding circuit 37. One channel is assigned to each of the transducer elements 20 in the ultrasound probe 101. The ultrasound probe 101 includes, with respect to each channel, the transmission delay circuit 32, the pulser 33, the transmission/reception switch 34, the low noise amplifier 35, and the time gain controller 36. The ultrasound probe 101 further includes, with respect to each of the sub-arrays 22, the transmission/reception control circuit 31 and the delay adding circuit 37. That is, the ultrasound probe 101 includes the electronic circuits illustrated in FIG. 3B for each of all of the sub-arrays 22 (forty two sub-arrays 22 in the example of FIG. 3A) in the main array 21. One or several ASICs 30 are included in the ultrasound probe 101.

The transmission/reception control circuit 31 controls transmission and reception of ultrasound. For example, the transmission/reception control circuit 31 receives a rate pulse output from the transmitter circuitry 110 and transmits the received rate pulse to the transmission delay circuit 32. The transmission/reception control circuit 31 receives the delay times of reflected wave signals output from the transmitter circuitry 110 and sets up the received delay times in the delay adding circuit 37 to be described later.

The transmission delay circuit 32 provides, to the rate pulse provided thereto from the apparatus main body 100, delay times corresponding to the respective transducer elements 20 and being needed for converging ultrasound generated from the transducer element 20 into a beam so that the transmission directivity can be determined. For example, the transmission delay circuit 32 provides, to the rate pulse provided thereto from the transmission/reception control circuit 31, delay times set up with respect to each channel, and outputs the rate pulse with the delay times to the pulser 33. The delay times to be provided to the rate pulse are controlled by the transmission/reception control circuit 31.

The pulser 33 generates a drive signal having a certain amplitude value. For example, the pulser 33 generates a drive signal at the timing based on the rate pulse output from the transmission delay circuit 32 and outputs to the transducer element 20. The amplitude value of the drive signal to be generated is controlled by the transmission/reception control circuit 31.

The transmission/reception switch 34 selectively switches, between the pulser 33 and the low noise amplifier 35, a component to which the transducer element 20 is connected. When the transmission/reception switch 34 is connected to the pulser 33, the transmission/reception switch 34 transmits, to the transducer element 20, a drive signal output from the pulser 33. When the transmission/reception switch 34 is connected to the low noise amplifier 35, the transmission/reception switch 34 outputs a reflected wave signal transmitted from the transducer element 20 to the low noise amplifier 35.

Here, the rate pulse with which the pulser 33 generates drive signals is derived from the transmitter circuitry 110. Reflected wave signals output to the low noise amplifier 35 are received by the receiver circuitry 120 as described later. That is, the transmission/reception switch 30 switches, among a plurality of alternatives including the transmitter circuitry 110 and the receiver circuitry 120, a component to which each of the transducer elements 20 included in the ultrasound probe 101 is connected. The transmission/reception switch 34 is an example of a switching circuit.

The low noise amplifier 35 receives a reflected wave signal from the transducer element 20 via the transmission/reception switch 34, then amplifies the received reflected wave signal with a previously determined gain, and then outputs the amplified reflected wave signal to the time gain controller 36.

Upon receiving the reflected wave signal transmitted from the low noise amplifier 35, the time gain controller 36 amplifies the reflected wave signal. The time gain controller 36 then outputs the amplified reflected wave signal to the delay adding circuit 37.

Upon receiving the reflected wave signal output for each channel from the time gain controller 36, the delay adding circuit 37 executes delay processing on the reflected wave signal for each channel. The delay processing is to give a delay amount needed for determining the reception directivity of the signal. The delay adding circuit 37 then executes addition processing of adding together the reflected wave signals for the respective channels on which the delay processing has been executed, and outputs a reflected wave signal obtained by the addition to the receiver circuitry 120 in the apparatus main body in the apparatus main body 100. This addition processing is performed on each channel within each of the sub-arrays 22. That is, the delay adding circuit 37 synthesizes (performs delay addition processing on) reflected wave signals for the respective channels within each of the sub-arrays 22.

As described so far, the ultrasound probe 101 according to the embodiment is a 2D array probe having a plurality of transducer elements 20 arrayed in a matrix. The ultrasound probe 101 includes the sub-arrays 22 each composed of a plurality of transducer elements 20, a main array 21 composed of the sub-arrays 22, and a delay adding circuit 37 configured to execute the delay addition processing with respect to each of the sub-arrays 22.

In some cases when the above-described 2D array probe is used, received echoes contain noise that occurs in a certain fixed timing pattern. For example, an analog switch is used for, in the processing performed by the delay adding circuit 37, repeatedly updating delay times for the respective channels that are stored in the delay memory. This analog switch causes noise (switch noise) when being switched. The analog switch is switched at a certain updating rate for updating delay times, noise attributable to the analog switch occurs in accordance with the updating rate and is sometimes contained as periodically occurring noise (also referred to as “periodic noise”) in received echoes.

In addition, as fixed noise, noise (also referred to as “fixed noise”) attributable to spurious transmission may be contained in received echoes. Here, spurious transmission means transmission of ultrasound at timing when the transmission is originally not permitted. For example, when switch noise that occurs when the transmission/reception switch 34 is switched from transmission to reception or vice versa is transmitted to the transducer element 20, transmission (spurious transmission) of weak ultrasound occurs even if it is originally not the right timing for ultrasound to be transmitted. In spurious transmission, ultrasound comes out as transmission waves from transducer elements in the same manner as proper ultrasound does, and consequently reflected in the interior of the subject P. In some cases, the reception echoes thus reflected are received by the transducer elements 20 and consequently contained as noise in proper reception echoes. Specifically, fixed noise attributed to spurious transmission occurs at switching from the transmission side to the reception side, and at switching from the reception side to the transmission side. The fixed noise therefore occurs before and after the proper received echoes, so that three overlapping signals appear.

As described so far, in some cases when a 2D array probe is used, received echoes contain periodic noise and fixed noise due to spurious transmission. Cyclical noise and fixed noise have fixed sizes, and are therefore considered to more likely appear when the amplitude of proper ultrasound to be transmitted is low. For example, in contrast harmonic imaging (CHI) in which micro bubbles are used as a contrast agent, the amplitude of proper ultrasound to be transmitted is set relatively low, fixed noise tends to appear.

With this point taken into account, the ultrasound diagnostic apparatus 1 according to this embodiment is configured to execute the following imaging method to which the pulse subtraction (PS)-tissue harmonic imaging (THI) technique is applied, in order to remove periodic noise and fixed noise.

That is, in the ultrasound diagnostic apparatus 1 according to the embodiment, the ultrasound probe 101 sequentially executes sets of ultrasound transmission respectively along a plurality of scan lines, the sets each being composed only of transmission of first ultrasound and transmission of second ultrasound, the first ultrasound having a first phase, the second ultrasound having a second phase that is 90 degrees different from the first phase. With respect to a first echo signal and a second echo signal that have been obtained via the ultrasound probe 101 and correspond to the first ultrasound and the second ultrasound, respectively, the calculation function 132 generates a subtraction signal by subtracting the second echo signal from the first echo signal. The image generating circuitry 150 generates an ultrasound image based on the subtraction signals generated for the respective scan lines. An imaging method employed in the ultrasound diagnostic apparatus 1 is described hereinbelow.

The embodiment hereinbelow describes, but is not limited to, processing of removing periodic noise and fixed noise that occur when a 2D array probe is used. For example, the above-described noise due to spurious transmission is noise that occurs also when the ultrasound probe 101 (for example, a 1D array probe) not being a 2D array probe is used. This means that this embodiment is effective in removing periodic noise and fixed noise when the ultrasound probe 101 that is not a 2D array probe is used.

Furthermore, the embodiment hereinbelow describes, but is not limited to, a case in which the THI technique is executed for extracting a second-order harmonic component. For example, the following part of the embodiment may be applied to the THI method for extracting a third- or higher-order harmonic component. The following part of the embodiment is not limited to being applied to the THI method and may be applied to the CHI method and may be broadly applied to imaging methods including fundamental waves.

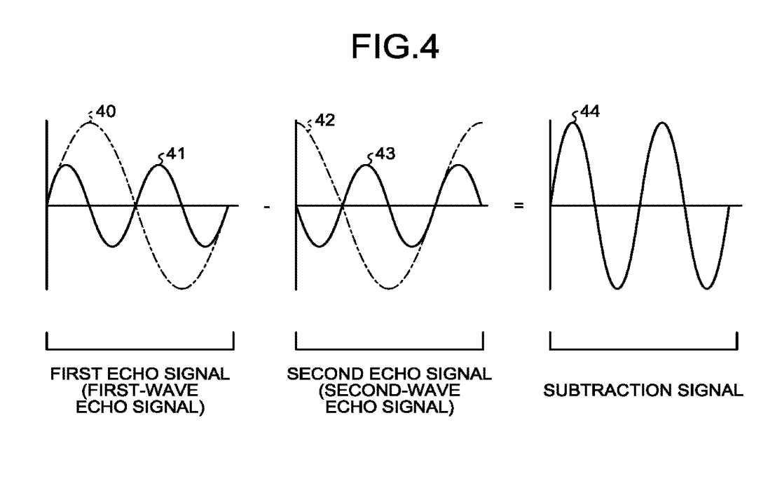

FIG. 4 is a diagram for explaining an imaging method employed in the ultrasound diagnostic apparatus 1 according to the embodiment. FIG. 4 illustrates processing of subtraction between echo signals from two times of acquisition that have been acquired in one set of ultrasound transmission composed of two times of transmission of ultrasound.

In FIG. 4, the waveform at the left represents the waveform of a first-wave echo signal (first echo signal) acquired by transmission of the first wave. The waveform at the center represents the waveform of a second-wave echo signal (second echo signal) acquired by transmission of the second wave. The waveform at the right represents the waveform of a subtraction signal. The example illustrated in FIG. 4 exemplifies a case in which the subtraction signal having the waveform at the right is obtained by subtracting the echo signal having the waveform at the center from the echo signal having the waveform at the left. With respect to each of the waveforms, the horizontal axis corresponds to time and the vertical axis corresponds to the amplitude.

For example, the transmitter circuitry 110 causes the ultrasound probe 101 to execute ultrasound scanning in accordance with a scan sequence set by the control circuitry 180. Specifically, the transmitter circuitry 110 causes the ultrasound probe 101 to execute ultrasound scanning in which sets of ultrasound transmission are sequentially executed respectively along a plurality of scan lines, the sets each being composed only of transmission of first ultrasound and transmission of second ultrasound, the first ultrasound having a first phase, the second ultrasound having a second phase that is substantially 90 degrees different from the first phase.

That is, the transmitter circuitry 110 causes the ultrasound probe 101 to execute, with respect to each of the scan lines within the scanning range, one set of ultrasound transmission composed of two times of transmission of ultrasound. For example, the transmitter circuitry 110 causes sine-wave ultrasound to be transmitted for transmission of the first wave (first transmission) of ultrasound (first ultrasound) and causes ultrasound (a cosine wave) obtained by rotating the phase of the first wave of ultrasound 90 degrees to be transmitted for transmission of the second wave (second transmission) of ultrasound (second ultrasound). As a result, the ultrasound probe 101 executes, sequentially for the respective scan lines, sets of ultrasound transmission in each of which: sine-wave ultrasound is transmitted for transmission of the first wave of ultrasound; and ultrasound obtained by rotating the phase of the first wave of ultrasound 90 degrees is transmitted for transmission of the second wave of ultrasound.

The receiver circuitry 120 then generates reflected wave data containing two echo signals for each of the scan lines. For example, the receiver circuitry 120 generates reflected wave data that contains: a first-wave echo signal acquired by transmission of the first wave of ultrasound; and a second-wave echo signal acquired by transmission of the second wave of ultrasound (refer to FIG. 4). The receiver circuitry 120 outputs the generated reflected wave data to the calculation function 132.

With respect to a first echo signal and a second echo signal that have been obtained via the ultrasound probe 101 and correspond to the first ultrasound and the second ultrasound, respectively, the calculation function 132 generates a subtraction signal by subtracting the second echo signal from the first echo signal. The calculation function 132 is an example of a calculation unit.

The above-described periodic noise and fixed noise are noise that is taken in at regular timing each time ultrasound is transmitted and received. That is, the periodic noise and the fixed noise are taken in at regular timing into both of the first-wave echo signal and the second-wave echo signal. With this point taken into account, the calculation function 132 subtracts, one time, the second-wave echo signal from the first-wave echo signal as illustrated in FIG. 4. In this manner, the calculation function 132 can remove the periodic noise and fixed noise that have been taken in into the first-wave echo signal and the second-wave echo signal at regular timing. The calculation function 132 then generates a subtraction signal by subtracting, one time, the second-wave based echo signal from the first-wave echo signal. The calculation function 132 generates subtraction signals for the respective scan lines, and outputs the generated subtraction signals corresponding to the respective scan lines to the filter function 133.

The filter function 133 applies a high-pass filter or a band-pass filter to the subtraction signals output from the calculation function 132. For example, the filter function 133 applies a filter that removes the fundamental-wave components contained in the subtraction signals corresponding to the respective scan lines to extract the second-order harmonic components. The filter function 133 is an example of a filter unit.

In the example illustrated in FIG. 4, a waveform 40 of a fundamental-wave component contained in the first-wave echo signal is expressed as sin θ. In this case, a waveform 41 of a second-order harmonic component contained in the first-wave echo signal is expressed as sin 2θ. A waveform 42 of a fundamental-wave component contained in the second-wave echo signal has a phase obtained by rotating the corresponding phase of the first-wave echo signal 90 degrees, and is therefore expressed as cos θ. In this case, a waveform 43 of a second-order harmonic component contained in the second-wave echo signal is expressed as −sin 2θ.

Here, the second-order harmonic components are considered. As a result of subtracting the waveform 43 of the second-order harmonic component corresponding to the second wave from the waveform 41 of the second-order harmonic component corresponding to the first wave, the amplitude of a waveform 44 of a second-order harmonic component contained in the subtraction signal is twice as large as the amplitude before the subtraction. Subsequently, for example, the filter function 133 applies a high-pass filter (or a band-pass filter) to the subtraction signal for each of the scan lines, thereby extracting the second-order harmonic component represented by the waveform 44 for the each. The filter function 133 outputs, to the B-mode data generation function 131, the subtraction signals for the respective scan lines that contain the extracted second-order harmonic components.

Consequently, the B-mode data generation function 131 generates B-mode data using the subtraction functions for the respective scan lines. The image generating circuitry 150 then generates ultrasound image data using the B-mode data generated by the B-mode data generation function 131. That is, the image generating circuitry 150 generates ultrasound image data based on the subtraction signals generated for the respective scan lines. Specifically, the image generating circuitry 150 generates the ultrasound image data using the second-order harmonic components extracted by the filter function 133.

Thus, the ultrasound diagnostic apparatus 1 according to the embodiment removes fixed noise and generates a subtraction signal having a second-order harmonic component twice as large (6 dB) by performing one subtraction. The content of FIG. 4 is merely an example and is not limiting. For example, ultrasound that is transmitted as the first wave is not limited to a sine wave, and may be any ultrasound that has any desired initial phase.

The difference in phase between ultrasound transmitted as the first wave and ultrasound transmitted as the second wave is not limited to 90 degrees and may be, for example, 270 degrees. In other words, the ultrasound probe 101 sequentially executes sets of ultrasound transmission respectively along a plurality of scan lines, the sets each being composed only of transmission of first ultrasound and transmission of second ultrasound, the first ultrasound having a first phase, the second ultrasound having a second phase that is substantially 90 degrees different from the first phase.

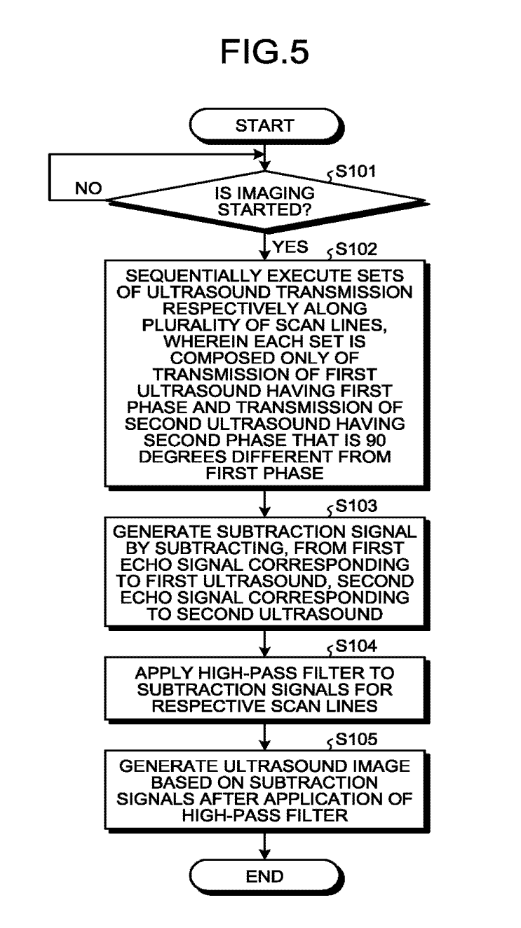

FIG. 5 is a flowchart for explaining a processing procedure employed in the ultrasound diagnostic apparatus 1 according to the embodiment. The processing procedure illustrated in FIG. 5 is started, for example, when an instruction indicating that imaging be started is received from the operator.

As illustrated in FIG. 5, for example, when the input device 102 receives, from the operator, an instruction that imaging be started (Yes at Step S101), the control circuitry 180 starts processing at Step S102 and subsequent steps. Before receiving the instruction indicating that imaging be started (No at Step S101), the control circuitry 180 does not start the following processing and stands by.

After imaging is started, the ultrasound probe 101 sequentially executes sets of ultrasound transmission respectively along a plurality of scan lines, the sets each being composed only of transmission of first ultrasound and transmission of second ultrasound, the first ultrasound having a first phase, the second ultrasound having a second phase that is 90 degrees different from the first phase (Step S102).

Subsequently, with respect to a first echo signal and a second echo signal that have been obtained via the ultrasound probe 101 and correspond to the first ultrasound and the second ultrasound, respectively, the calculation function 132 generates a subtraction signal by subtracting the second echo signal from the first echo signal (Step S103). For example, the calculation function 132 performs a subtraction of an echo signal of the first wave minus an echo signal of the second wave, thereby generating a subtraction signal as a result of removal of fixed noise.

The filter function 133 applies a high-pass filter to the subtraction signals for the respective scan lines (Step S104). For example, the filter function 133 uses the high-pass filter to remove fundamental-wave components contained in the subtraction signals for the respective scan lines, thereby extracting the second-order harmonic components.

The image generating circuitry 150 then generates an ultrasound image based on the subtraction signals generated for the respective scan lines (Step S105). For example, the image generating circuitry 150 generates ultrasound image data using B-mode data generated from the subtraction signals by the B-mode data generation function 131.

If it is desired to perform substantially real-time imaging, the ultrasound diagnostic apparatus 1 repeatedly executes processing from Step S102 to Step S105 to generate substantially real-time ultrasound image data and displays the data. Thereafter, when an operation indicating that the imaging be ended is received from the operator, the ultrasound diagnostic apparatus 1 ends the processing procedure of FIG. 5.

The content of FIG. 5 is merely an example and is not limiting. For example, the processing at Step S104 need not necessarily be executed. In such a case, the second-order harmonic components are not extracted, the subtraction signals containing the fundamental wave signals are used for generating ultrasound image data.

Otherwise, for example, the processing at Step S104 may be executed before the processing at Step S103. In such a case, the filter function 133 applies a high-pass filter to reflected wave data that contains the first echo signals and the second echo signals to remove fundamental-wave components contained in the first echo signals and the second echo signals, thereby extracting second-order harmonic components. The calculation function 132 then generates subtraction signals using the extracted second-order harmonic components of the first echo signals and the second echo signals.

Thus, the ultrasound diagnostic apparatus 1 according to the embodiment can remove noise by executing an imaging method to which the PS-THI technique is applied.

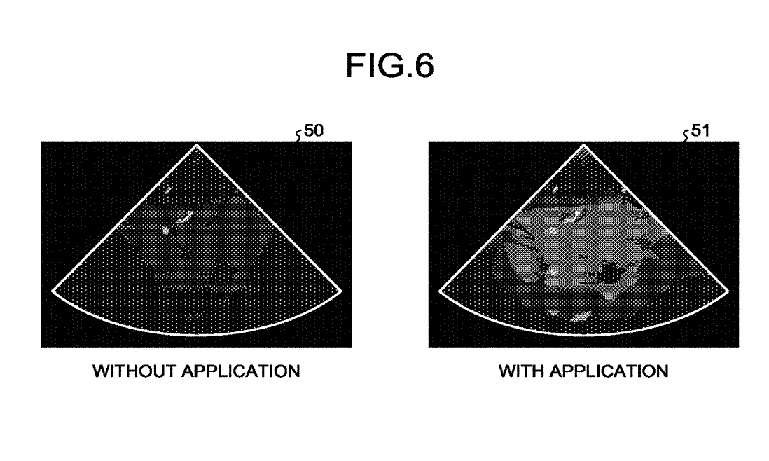

FIG. 6 is a diagram for explaining effects that are produced by the ultrasound diagnostic apparatus 1 according to the embodiment. While the left illustration in FIG. 6 represents an ultrasound image 50 obtained without the application of the imaging method employed in the ultrasound diagnostic apparatus 1, the right illustration in FIG. 6 represents an ultrasound image 51 obtained with the application of the imaging method employed in the ultrasound diagnostic apparatus 1. The ultrasound image 50 and the ultrasound image 51 are images that have noise levels adjusted to be the same.

In the ultrasound image 51 obtained by the ultrasound diagnostic apparatus 1 with the imaging method applied, the amplitude of a second-order harmonic component included in each subtraction signal is “sin 2θ−(−sin 2θ)=2 sin 2θ”, which is twice as large as the amplitude before the subtraction. As a result, as illustrated in FIG. 6, the ultrasound image 51 is presented with higher brightness than the ultrasound image 50 with the noise levels thereof adjusted to be the same. That is, it is found that the imaging method employed in the ultrasound diagnostic apparatus 1 has resulted in a 6-dB improvement in signal-to-noise (SN) ratio (20×log 2=6). Although not illustrated, if the signal levels of the ultrasound image 50 and the ultrasound image 51 are adjusted to be the same, the ultrasound image 51 actually can be obtained with a gain that is 6 dB smaller than a gain with which the ultrasound image 50 is obtained. That is, it can be concluded that the imaging method employed in the ultrasound diagnostic apparatus 1 has resulted in a 6-dB improvement in signal-to-noise (SN) ratio.

### Other Embodiments

Embodiments other than the above-described embodiment can be implemented in various different forms.

### Amplitude Modulation

For example, the above embodiment explains a case in which a set of ultrasound scanning composed of two times of ultrasound transmission between which the phase is modulated. Embodiments are, however, not limited to this case. For example, processing similar to the above processing can be executed in a case where a set of ultrasound scanning composed of two times of ultrasound transmission between which the amplitude is modulated.

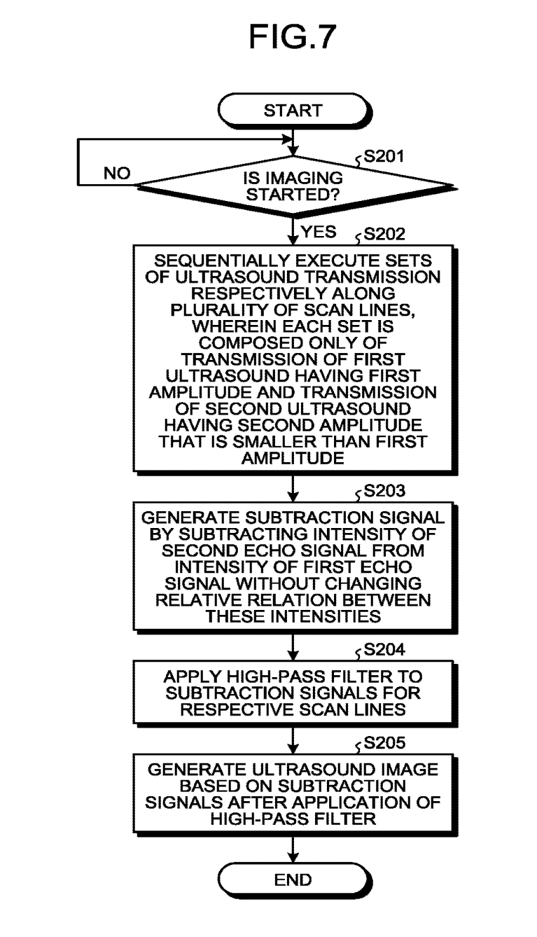

FIG. 7 is a flowchart for explaining a processing procedure employed in an ultrasound diagnostic apparatus 1 according to another embodiment. The processing procedure illustrated in FIG. 7 is started, for example, when an instruction indicating that imaging be started is received from the operator.

As illustrated in FIG. 7, for example, when the input device 102 receives, from the operator, an instruction indicating that imaging be started (Yes at Step S201), the control circuitry 180 starts processing at Step S202 and subsequent steps. Before receiving the instruction indicating that imaging be started (No at Step S201), the control circuitry 180 does not start the following processing and stands by.

After imaging is started, the ultrasound probe 101 sequentially executes sets of transmission respectively along a plurality of scan lines, the sets each being composed only of transmission of first ultrasound and transmission of second ultrasound, the first ultrasound having a first amplitude, the second ultrasound having a second amplitude that is smaller than the first amplitude (Step S202). For example, the transmitter circuitry 110 causes the ultrasound probe 101 to execute ultrasound scanning in which: amplitude modulation is performed; and a set of ultrasound transmission is executed with respect to each scan line, the set being composed only of transmission of first ultrasound and transmission of second ultrasound, the second ultrasound having an amplitude smaller than the amplitude of the first ultrasound.

Subsequently, without changing the relative relation between the intensity of a first echo signal corresponding to the first ultrasound and the intensity of a second echo signal corresponding to the second ultrasound, the calculation function 132 generates a subtraction signal by subtracting the intensity of the second echo signal from the intensity of the first echo signal (Step S203). For example, the calculation function 132 performs the subtraction while keeping the relative relation between the intensity of the first echo signal and the intensity of the second echo signal unchanged. This is different from typical amplitude modulation methods such that either of the intensity of the first echo signal and the intensity of the second echo signal is changed and such that the intensities are modulated with different multiplying factors. That is, the subtraction is performed without the intensity of the first echo signal and the intensity of the second echo signal modulated. If these intensities are modulated, the subtraction is performed after the intensities are modulated with the same multiplying factor. In this manner, the calculation function 132 generates a subtraction signal from which fixed noise has been removed.

The filter function 133 then applies a high-pass filter to the subtraction signals corresponding to the respective scan lines (Step S204). For example, the filter function 133 uses the high-pass filter to remove fundamental-wave components contained in the subtraction signals for the respective scan lines, thereby extracting the second-order harmonic components.

The image generating circuitry 150 then generates an ultrasound image based on the subtraction signals generated for the respective scan lines (Step S205). For example, the image generating circuitry 150 generates ultrasound image data using B-mode data generated from the subtraction signals by the B-mode data generation function 131.

The content of FIG. 7 is merely an example and is not limiting. For example, the processing at Step S204 need not necessarily be executed. Otherwise, for example, the processing at Step S204 may be executed before the processing at Step S203.

### Use of Third- or Higher-Order Harmonic Components

For example, while the above embodiment explains an imaging method using a second-order harmonic components, embodiments are not limited to this imaging method, and third- or higher-order harmonic components may be used.

For example, the ultrasound probe 101 executes, sequentially for the respective scan lines, sets of ultrasound transmission each composed of transmission of the first wave of ultrasound and transmission of the second wave of ultrasound obtained by rotating the phase of the first wave of ultrasound 60 degrees. In this case, the waveform of a third-order harmonic component contained in the first-wave echo signal is expressed as sin 3θ. The waveform of a third-order harmonic component contained in the second-wave echo signal has a phase obtained by rotating the corresponding phase of the first-wave echo signal 60 degrees, and is therefore expressed as −sin 3θ.

The calculation function 132 then generates a subtraction signal by subtracting the second-wave echo signal from the first-wave echo signal. In this case, the amplitude of a third-order harmonic component therein is “sin 3θ−(−sin 3θ)=2 sin 3θ”, which is twice as large as the amplitude before the subtraction. In this manner, the ultrasound diagnostic apparatus 1 can remove fixed noise and obtain the sensitivity of a harmonic component. That is, in the ultrasound diagnostic apparatus 1, the filter function 133 extracts second- or higher-order harmonic components.

### Subtraction Processing Using White Noise

For example, while the above embodiments explain cases in which one set of ultrasound scanning composed of two times of ultrasound transmission, embodiments are not limited to these cases. For example, acquisition of white noise may be performed instead of the second ultrasound transmission and followed by subtraction processing.

That is, the ultrasound probe 101 transmits an ultrasound wave, and then receives an echo signal corresponding to the ultrasound wave. Subsequently, without transmitting an ultrasound wave, the ultrasound probe 101 receives a noise signal representing white noise by performing the same reception processing as processing of receiving an echo signal. This noise signal contains ordinary white noise, and inevitably contains periodic noise attributed to the delay addition processing performed by the 2D array probe.

The calculation function 132 then generates a subtraction signal by subtracting the noise signal from the echo signal generated by the ultrasound probe 101. In this manner, the ultrasound diagnostic apparatus 1 can remove periodic noise from the echo signal.

### Application to Ultrasound Diagnosis Apparatus Employing Highly Functional Ultrasound Probe

In recent years, a technique has been known that enables the main functions relating to transmission and reception of ultrasound to be incorporated into the housing of an ultrasound probe and enables an ultrasound diagnostic apparatus to be constructed by connecting this ultrasound probe (hereinafter referred to as “highly functional ultrasound probe”) to a versatile information processing apparatus such as a personal computer or a tablet terminal. The above-described embodiments can be applied to an ultrasound diagnostic apparatus employing the highly functional ultrasound probe.

That is, the highly functional ultrasound probe includes various circuits (for example, the transmitter circuitry 110 and the receiver circuitry 120) provided for implementing the main functions relating to transmission and reception of ultrasound. For this reason, periodic noise and fixed noise are highly likely to occur in association with actuation of these circuits. Thus, periodic noise and fixed noise that occur in the highly functional ultrasound probe can be removed by the application of the configurations according to the above-described embodiments.

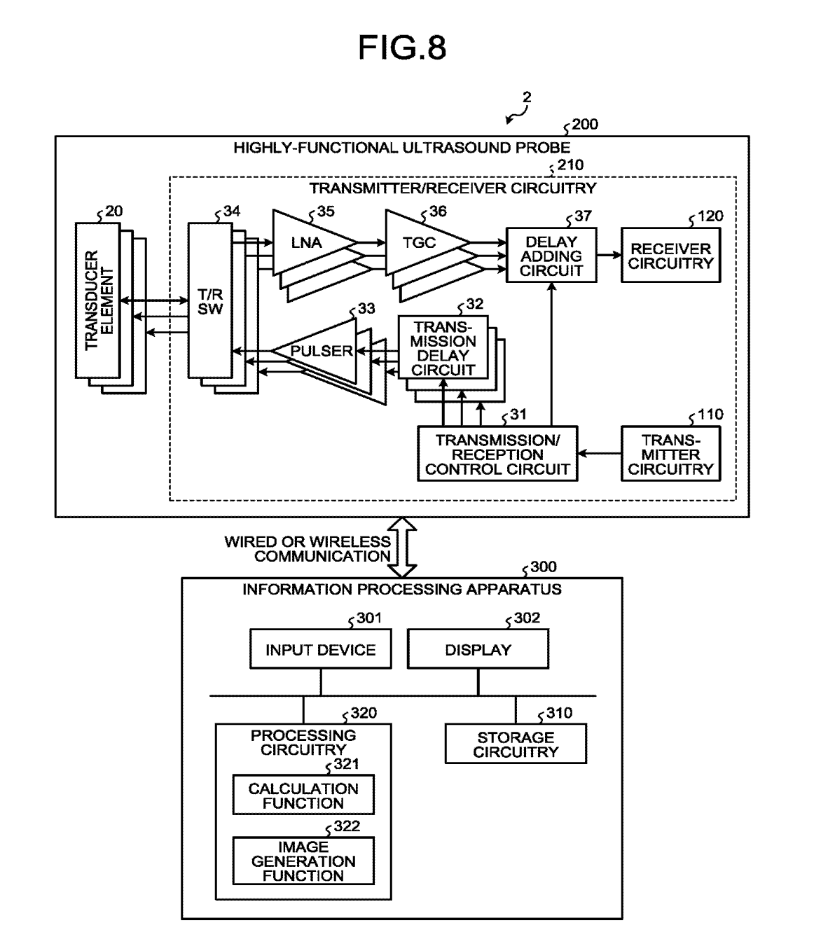

FIG. 8 is a block diagram illustrating an exemplary configuration of the ultrasound diagnostic apparatus 2 according to still another embodiment. As illustrated in FIG. 8, the ultrasound diagnostic apparatus 2 according to this other embodiment includes a highly functional ultrasound probe 200 and an information processing apparatus 300. The highly functional ultrasound probe 200 and the information processing apparatus 300 are connected to each other by wired or wireless communication.

The highly functional ultrasound probe 200 includes a plurality of transducer elements 20 and a transmitter/receiver circuitry 210. This transmitter/receiver circuitry 210 includes a transmission/reception control circuit 31, the transmission delay circuits 32, the pulsers 33, the transmission/reception switches 34, the low noise amplifiers 35, the time gain controllers 36, the delay adding circuit 37, a transmitter circuitry 110, and the receiver circuitry 120. The transmission/reception control circuit 31, the transmission delay circuits 32, the pulsers 33, the transmission/reception switches 34, the low noise amplifiers 35, the time gain controllers 36, and the delay adding circuit 37 that are illustrated in FIG. 8 perform basically the same processing as the transmission/reception control circuit 31, the transmission delay circuits 32, the pulsers 33, the transmission/reception switches 34, the low noise amplifiers 35, the time gain controllers 36, and the delay adding circuit 37 that are illustrated in FIG. 3B. Therefore, the same reference signs are assigned to these components, and descriptions thereof are omitted here. The transmitter circuitry 110 and the receiver circuitry 120 that are illustrated in FIG. 8 perform basically the same processing as the transmitter circuitry 110 and the receiver circuitry 120 that are illustrated in FIG. 1. Therefore, the same reference signs are assigned to these components, and descriptions thereof are omitted here.

The information processing apparatus 300 is for example, a versatile apparatus such as a personal computer or a tablet terminal. The information processing apparatus 300 includes an input device 301, a display 302, storage circuitry 310, and processing circuitry 320. The input device 301, the display 302, the storage circuitry 310, and the processing circuitry 320 are communicably connected to one another.

The input device 301 is an input device, such as a mouse, a keyboard, or a touch panel, provided for receiving various instructions and setting requests from the operator. The display 302 is a display device that displays thereon medical images and displays thereon a GUI that the operator uses for inputting various setting requests using the input device 301.

The storage circuitry 310 is, for example, a not-AND (NAND) flash memory or a hard disk drive (HDD), and stores therein computer programs to be used for displaying medical image data and GUIs and information to be used by the computer programs.

The processing circuitry 320 is electronic equipment (a processor) that controls the entire processing in the information processing apparatus 300. The processing circuitry 320 executes a calculation function 321 and an image generating function 322. The calculation function 321 performs basically the same processing as the calculation function 132 illustrated in FIG. 2. The image generating function 322 performs basically the same processing as the image generating circuitry 150 illustrated in FIG. 1. For example, the calculation function 321 and the image generating function 322 have been stored in the storage circuitry 310 while taking forms of computer-executable programs. The processing circuitry 320 loads and executes the programs, thereby implementing the functions (the calculation function 321 and the image generating function 322) that correspond to the respective programs that have been loaded. Although not illustrated, in the information processing apparatus 300, the same functions as those of the B-mode processing circuitry 130 and the Doppler processing circuitry 140 illustrated in FIG. 1 are implemented.

That is, the highly functional ultrasound probe 200 sequentially executes sets of ultrasound transmission respectively along a plurality of scan lines, the sets each being composed only of transmission of first ultrasound and transmission of second ultrasound, the first ultrasound having a first phase, the second ultrasound having a second phase that is substantially 90 degrees different from the first phase. In addition, with respect to the first echo signal and the second echo signal that have been obtained via the highly functional ultrasound probe 200 and correspond to the first ultrasound and the second ultrasound, respectively, the calculation function 321 generates a subtraction signal by subtracting the second echo signal from the first echo signal. The image generating function 322 then generates an ultrasound image based on the subtraction signals generated for the respective scan lines.

In this manner, the ultrasound diagnostic apparatus 2 can remove noise as with the ultrasound diagnostic apparatus 1. That is, the foregoing embodiments can remove periodic noise and fixed noise not only when being applied to a 2D array probe but also, for example, when being applied to the ultrasound diagnostic apparatus 2 employing the highly functional ultrasound probe 200.

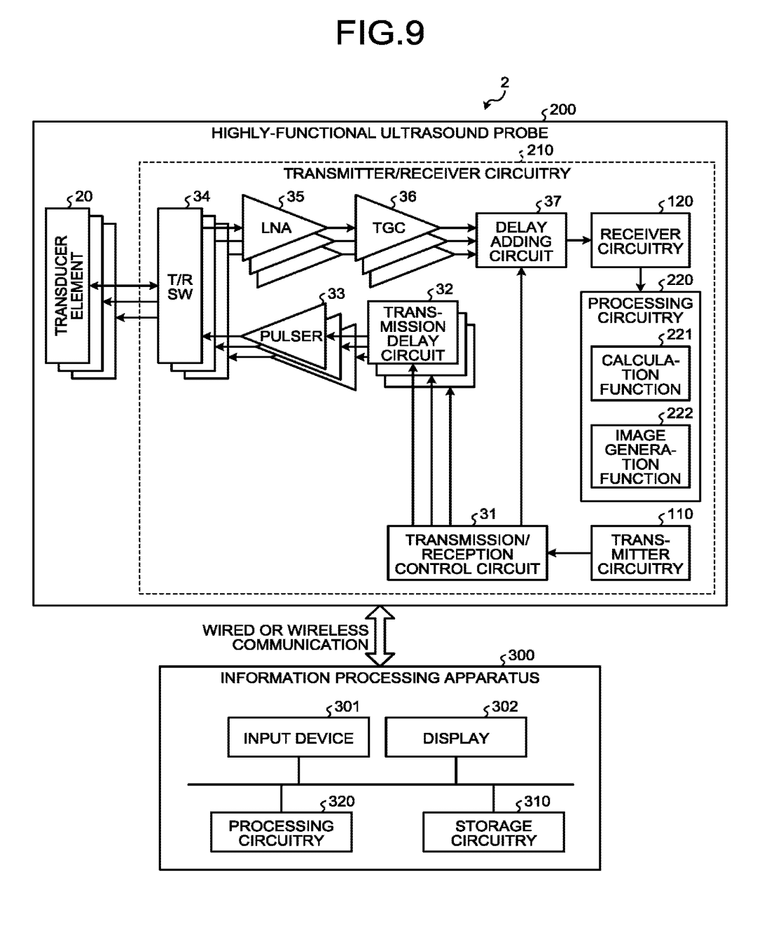

FIG. 8 merely illustrates an example, and the content illustrated in FIG. 8 is not limiting. For example, FIG. 8 exemplifies a case in which it is on the information processing apparatus 300 that images are produced from reflected wave data acquired by the highly functional ultrasound probe 200. Embodiments are, however, not limited to this case. For example, an embodiment may be configured so that images may be produced on the highly functional ultrasound probe 200 and that image data may be transmitted from the highly functional ultrasound probe 200 to the information processing apparatus 300. For example, as illustrated in FIG. 9, the highly functional ultrasound probe 200 includes processing circuitry 220. The processing circuitry 220 includes the calculation function 221 and the image generating function 222. In the highly functional ultrasound probe 200, the calculation function 221 generates a subtraction signal by subtracting the second echo signal corresponding to the second ultrasound from the first echo signal corresponding to the first ultrasound. Also in the highly functional ultrasound probe 200, the image generating function 222 then generates an ultrasound image based on the subtraction signals generated for the respective scan lines. The highly functional ultrasound probe 200 then transmits, to the information processing apparatus 300, the ultrasound image after noise is removed therefrom. The ultrasound diagnostic apparatus 2 illustrated in FIG. 9 can reduce a data amount transmitted from the highly functional ultrasound probe 200 to the information processing apparatus 300 by comparison to configurations illustrated in FIG. 8. Therefore, the ultrasound diagnostic apparatus 2 illustrated in FIG. 9 can reduce a risk that a transmission from the highly functional ultrasound probe 200 to the information processing apparatus 300 is not possible.

The term “processor” used in the above description means, for example, a circuit such as a central processing unit (CPU), a graphics processing unit (GPU), an application specific integrated circuit (ASIC), or a programmable logic device (examples of which include a simple programmable logic device (SPLD), a complex programmable logic device (CPLD), and a field programmable gate array (FPGA)). The processor implements a function by reading out and executing a program stored in storage circuitry. Instead of being stored in a storage circuitry 170, the program may be configured to be embedded directly in a circuit of the processor. In this case, the processor implements a function by reading out and executing the computer program embedded in the circuit. Each processor in the present embodiments is not limited to being configured as a single circuit for that individual processor, and may be configured as a plurality of independent circuits combined into one processor to implement the function thereof. Furthermore, a plurality of components in FIG. 1 may be integrated into one processor to implement the functions thereof.

Of the respective steps of processing described in the above embodiments, the whole or a part of those described as being to be automatically performed can be manually performed, or the whole of a part of those described as being to be manually performed can be automatically performed by known methods. In addition, the processing procedures, the control procedures, the specific names, and the information including various kinds of data and parameters described herein and illustrated in the drawings can be changed as desired unless otherwise specified.

The ultrasound imaging method described in the above embodiments can be implemented by executing a previously prepared ultrasound imaging program on a computer such as a personal computer or a workstation. This ultrasound imaging method can be distributed via a network such as the Internet. The ultrasound imaging method can also be recorded on a computer-readable recording medium such as a hard disk, a flexible disk (FD), a compact disc read only memory (CD-ROM), a magnetic optical disc (MO), or a digital versatile disc (DVD) to be executed by being loaded from the recording medium by the computer.

According to at least one of the embodiments described above, noise can be removed.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An ultrasound diagnostic apparatus comprising:
an ultrasound probe configured to sequentially execute sets of ultrasound transmission respectively along a plurality of scan lines, the sets each being composed only of transmission of first ultrasound and transmission of second ultrasound, the first ultrasound having a first phase, the second ultrasound having a second phase that is substantially 90 degrees different from the first phase; and
processing circuitry configured to
with respect to a first echo signal and a second echo signal that have been acquired via the ultrasound probe and correspond to the first ultrasound and the second ultrasound, respectively, generate a subtraction signal by subtracting the second echo signal from the first echo signal, and
generate an ultrasound image based on the subtraction signals generated for the scan lines.

2. The ultrasound diagnostic apparatus according to claim 1, further comprising a filter configured to apply a high-pass filter or a band-pass filter to the first echo signals and the second echo signals, or to the subtraction signals.

3. The ultrasound diagnostic apparatus according to claim 2, wherein
the filter applies a filter that removes fundamental-wave components contained in the first echo signals and the second echo signals or in the subtraction signals to extract at least second-order harmonic components, and
the processing circuitry generates the ultrasound image using the extracted second-order harmonic components.

4. The ultrasound diagnostic apparatus according to claim 1, further comprising a switching circuit configured to select, from among a plurality of alternative units including transmitter circuitry and receiver circuitry, a unit to which each of the transducer elements included in the ultrasound probe is connected.

5. The ultrasound diagnostic apparatus according to claim 1, wherein the ultrasound probe is a 2D array probe having a plurality of transducer elements arrayed in a matrix.

6. The ultrasound diagnostic apparatus according to claim 5,
wherein the 2D array probe comprises:
a plurality of sub-arrays each composed of a plurality of transducer elements;
a main array composed of the sub-arrays; and
a delay adding circuit configured to execute processing of adding together delays of echo signals with respect to each of the sub-arrays.

7. An ultrasound imaging method comprising:
causing an ultrasound probe to sequentially execute sets of ultrasound transmission respectively along a plurality of scan lines, the sets each being composed only of transmission of first ultrasound and transmission of second ultrasound, the first ultrasound having a first phase, the second ultrasound having a second phase that is substantially 90 degrees different from the first phase;
with respect to a first echo signal and a second echo signal that have been acquired via the ultrasound probe and correspond to the first ultrasound and the second ultrasound, respectively, generating a subtraction signal by subtracting the second echo signal from the first echo signal; and
generating an ultrasound image based on the subtraction signals generated for the scan lines.
